(19) United States
(12) Patent Application Publication
(10) Pub. No.: US 2024/0304338 A1
(43) Pub. Date: Sep. 12, 2024

(54) STABILITY DATA MANAGEMENT SYSTEM FOR PHARMACEUTICAL COMPANIES

(71) Applicant: Navneet Kumar, Lexington, MA (US)

(72) Inventor: Navneet Kumar, Lexington, MA (US)

(73) Assignee: Myra EB Systems, Boxborough, MA (US)

(21) Appl. No.: 18/614,703

(22) Filed: Mar. 24, 2024

Related U.S. Application Data
(60) Provisional application No. 63/489,106, filed on Mar. 8, 2023.
(60) Provisional application No. 63/490,247, filed on Mar. 15, 2023.
(60) Provisional application No. 63/490,509, filed on Mar. 15, 2023.

(51) Int. Cl. G16H 70/40; G16H 10/00

(52) U.S. Cl. CPC G16H 70/40; G16H 10/00

(57) ABSTRACT

The data management system allows for users to input data into a centralized cloud based platform allowing for greater ease of access. The system also provides powerful analysis tools designed to give visualizations for any drug process and stability data the user wishes to graph. AI/ML can further help with Yield optimizations, process control, and ability to quick review of stability data.

### FIELD OF INVENTION

The amount of data needed to be aggregated, organized, and analyzed in the pharmaceutical industries is massive and therefore, a specially designed IT system that handles Stability Data Management and a plurality of Stability Studies is needed to present the information to the users in a clear and simple way, to enable decisions to be made based on the available information. The industry is in need of an IT system that keeps data organized as well as create critical trend analyses quickly and provide them to the users making the decisions based on the available data.

### BACKGROUND INFORMATION

In the industry, the current systems need to capture the data and delineate it by Product, Batch, and Unit Operation for the In-Process Data between batches, which could be process development runs, pilot runs, engineering runs, and commercial runs. The current systems extract this data through integration with the PI system, LIMS lab system, ELN, and also excel templates are available for easy data entry. For the Stability and Release of Data, the system can pull data from Data Lakes and LIMS systems. The systems can also attach files typically associated with the Release and Stability data, where the specification for each product include three standard deviations regarding the control limits. The aggregate data is used for creating process control charts and stability trends, and based on the in-process data for a unit operation. Therefore an AI/ML engine is needed that can predict the ideal case for the rest of the batch data, where the E.gBased on the early growth parameters for the N-3 stage can expect the VCD and Cell Count at the Bioreactor stage. There is also a need for a system that can generate various correlations and graphical trends based on customer needs based on the Bio-reactor data, the insights can predict the Yield Data at the downstream steps and the Drug Substance Release Date. Based on the Stability data, the system can predict the shelf life more efficiently and the AI/ML engine(s) can scan through pictures and analytical data for a given assay such as SEC, Peptide mapping, and sequencing to provide insights around passing and failing of the assay and if the procedure is correctly followed.

### SUMMARY OF INVENTION

This application claims benefits from provisional patent applications 63/489,106, 63/490,247, and 63/490,509 and these applications are incorporated in their entirety into this application. The invention can be implemented in two versions, an Enterprise version and a Professional version. In both versions, the data is captured by Product, Batch, and Unit Operation for the In-Process Data between batches of different drugs, medications, vaccines, etc. The batches could be process development runs, pilot runs, engineering runs, and commercial runs. Modules can extract this data through integration with the PI system, LIMS lab system, ELN, and also excel templates are available for easy data entry, and for the Stability and Release of Data, the system can pull from Data Lakes and LIMS systems. The system can also attach files typically associated with the Release and Stability data, and based on the in-process data for a unit operation the system can enter the specification for each product for three standard deviations regarding the control limits. The aggregate data is then used for creating process control charts and stability trends and an AI/ML engine can predict the ideal case for the rest of the batch data. E.gBased on the early growth parameters for the N-3 stage, one can expect the VCD and Cell Count at the Bioreactor stage. The system can generate various correlations and graphical trends based on customer needs. Based on the Bio-reactor data, generated insights can predict the Yield Data at the downstream steps and the Drug Substance Release Date, and based on the Stability data, the system can predict the shelf life more efficiently. The AI/ML engine(s) can scan through pictures and analytical data for a given assay such as SEC, Peptide mapping, and sequencing to provide insights around passing and failing of the assay and if the procedure is correctly followed. The data management system allows users to input data into a centralized cloud-based platform, allowing greater ease of access. The system also provides powerful analysis tools to visualize any drug data the user wishes to graph. Key things using AI/ML are: Process control using real-time process data, data fusion by collating data from various data sources, and Predictions and Yield optimization using the engine. Addition AI/ML also provides Predictive maintenance for key equipment such as pumps and mixers using an algorithm.

This is a specially designed IT system that handles stability data management and stability studies in pharmaceutical industries. The industry currency needs a system that keep data organized and can quickly produce critical trends which are easy to interpret for a user. The large amount of data to be analyzed lies in various folders and is challenging to assemble for final analysis. This application is designed very specifically with keeping in mind the pharmaceutical industry.

### BRIEF DESCRIPTIONS OF FIGURES

FIG. 1 shows an overview of the system.

FIG. 2 shows a login interface.

FIG. 3 shows an Add Product interface.

FIG. 4 shows a Batches interface.

FIG. 5 shows an Add Specification interface.

FIG. 6 shows an interface for adding additional information about a pharmaceutical.

FIG. 7 shows an Add Variable interface.

FIG. 8 shows a Study List interface.

FIG. 9 shows a Study interface.

FIG. 10 shows a Study interface.

FIG. 11 shows an example variable that is Capture Cycle Yield interface.

FIG. 12 shows a Variable interface.

FIG. 13 shows an Add Data interface.

FIG. 14 shows an Add Perform Study interface.

FIG. 15 shows an interface for adding additional information about a pharmaceutical.

FIG. 16 shows an interface for adding additional information about a pharmaceutical.

FIG. 17 shows a chart that indicates trends.

FIG. 18 shows a chart that indicates trends.

FIG. 19 shows an interface for Study/Test Results.

FIG. 20 shows an interface for Study/Test Results.

FIG. 21 shows an interface for Study/Test Results.

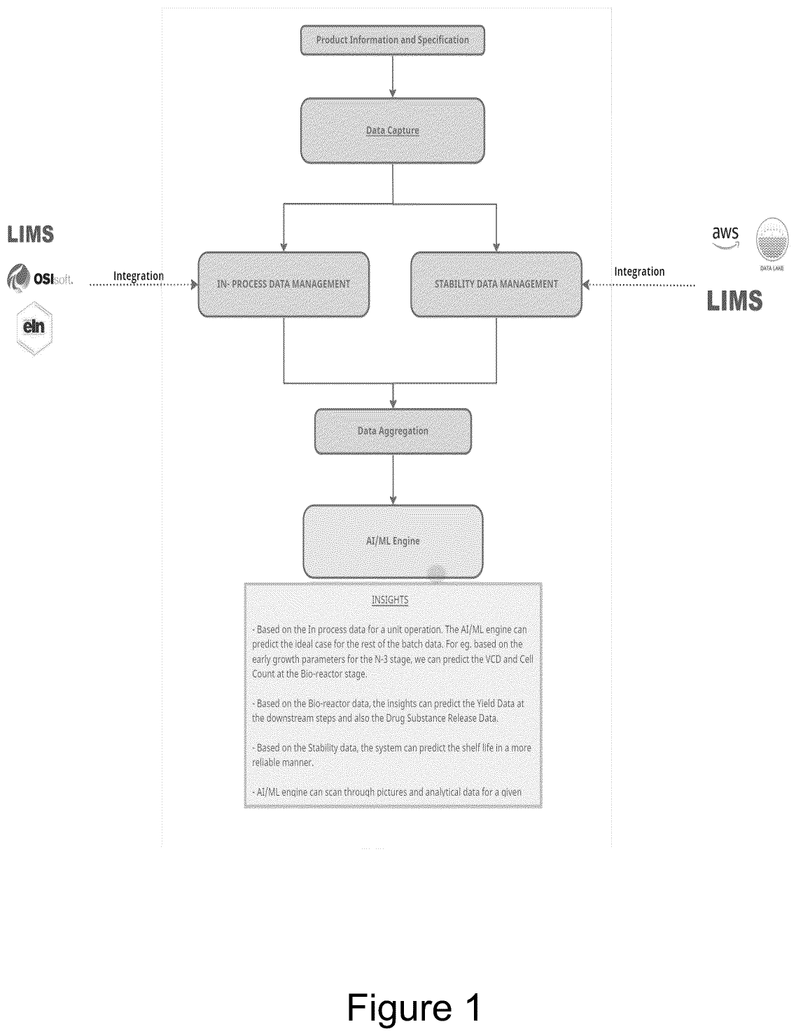

As seen in the system shown in FIG. 1, the data is captured by Product, Batch, and Unit Operation for the In-Process Data between batches. The batches could be: process development runs, pilot runs, engineering runs, and commercial runs. The module can extract this data through integration with the PI system, LIMS lab system, ELN, and also excel templates that are available for easy Data Entry. For the Stability and Release of Data, the system can pull from Data Lakes and LIMS systems. The system can also attach files typically associated with the Release and Stability data. The system can enter the specification for each product for three standard deviations regarding the control limits. The aggregate data is used for creating process control charts and stability trends. Based on the in-process data for a unit operation. The AI/ML engine can predict the ideal case for the rest of the batch data. E.gBased on the early growth parameters for the N-3 stage, we can expect the VCD and Cell Count at the Bioreactor stage. The system can generate various correlations and graphical trends based on customer needs. Based on the Bio-reactor data, the insights can predict the Yield Data at the downstream steps and the Drug Substance Release Date. Based on the Stability data, the system can predict the shelf life more efficiently. AI/ML engine can scan through pictures and analytical data for a given assay such as SEC, Peptide mapping, and sequencing to provide insights around passing and failing of the assay and if the procedure is correctly followed.

In the professional version, the high-level functionality and use cases in the application are explained to the user. Users can be of two types, Users with Edit access and User with View Only access. A user with edit access can create DS or DP, make batches, save the results of the tests performed, and check the trends. At the same time, a user with view-only access can only check the test results and trends. Registered users can log in using their valid credentials to access the application. Users can use the ‘Forgot Password’ feature if they have forgotten the password. Users can check the trends by selecting specific tests and timelines. Directions can be seen based on the test results fed into the system. Users can select different options to check the variety of trends with the associated data in the system and both types of users can access this functionality. The user can select DS or DP and a specific batch and timeline to check the results associated with that particular DS or DP batch timeline. Both types of users can access this functionality. Results are generally shown in Tabular format but may vary according to the test type. There can be multiple result values for a single test. The user can create a new DS or DP in the system by entering all the relevant data required to define a DS or DP. The user can then create a batch for any declared DS or DP by adding all the relevant information required to define a Batch. A test form must be created before a user can perform the test and enter the results into the system. Test forms usually belong to a specific test for a particular timeline and batch for a specific DS or DP. While creating the test form, the user can make changes to the test details, acceptance criteria, etc., and then save it so that it can be used to feed the test results into the system.

In the Enterprise Version, the first step is for a user of the data management system to login through the URL provided using their email and password. If the user selects the Batches option, this will bring the user to the page where they can enter the batch ID, location as well as description, if necessary. The user can choose from any drug substance/product that has been entered into the system in the previous step to create a new batch.

The dropdown menus will only activate when either drug product or substance has been selected. The user can use the tab called Add Variables to add different variables to the batch. Here the user can add any variables that they would like to track by adding the name of the variable, the unit data type or unit operations (Such as protein A or bioreactor), and a description of the variable, as well as the response type. To determine the response type, the user can select one of the following input options: Text, Input, or Dropdown. When the Text option is selected, the Lower and Upper range specification when creating a study can be entered. The Input option allows for creating a Label specification when designing a study. The Drop Down option allows for multiple possibilities to inputted when creating a study.

To create a new specification, the user must select the option called Specifications. By clicking on the blue plus sign on the top right of the screen, the user is brought to a page where they can enter a new study. Enter the drug as well as the type of Study by selecting off of the dropdown that opens when clicked on (if stable, the user will be prompted to name the Study as well). Below is a list of the variables that have been entered into the system. The user can select what the Study measures by clicking the boxes, and by clicking on the variable name, the user can set the details for the variables for each Study. The information needed will vary depending on the study type and the system can help the user determine what type of variables need to be entered. The user can select the Trends option to view the trends and fill in the information requested from each dropdown menu trend type, Drug, Batch, Study, timeline, unit type, and which variable. The user can input multiple studies within the same batch to visually represent how a study affects the variable across the same batch and view the corresponding trends within a batch. The user can select multiple batches so as to input various sets and a single study to create a visualization of how the Study affects the variable across multiple batches. The user can input numerous batches across a single timeline and single Study, this will enable the user to visualize how the variable affects different sets.

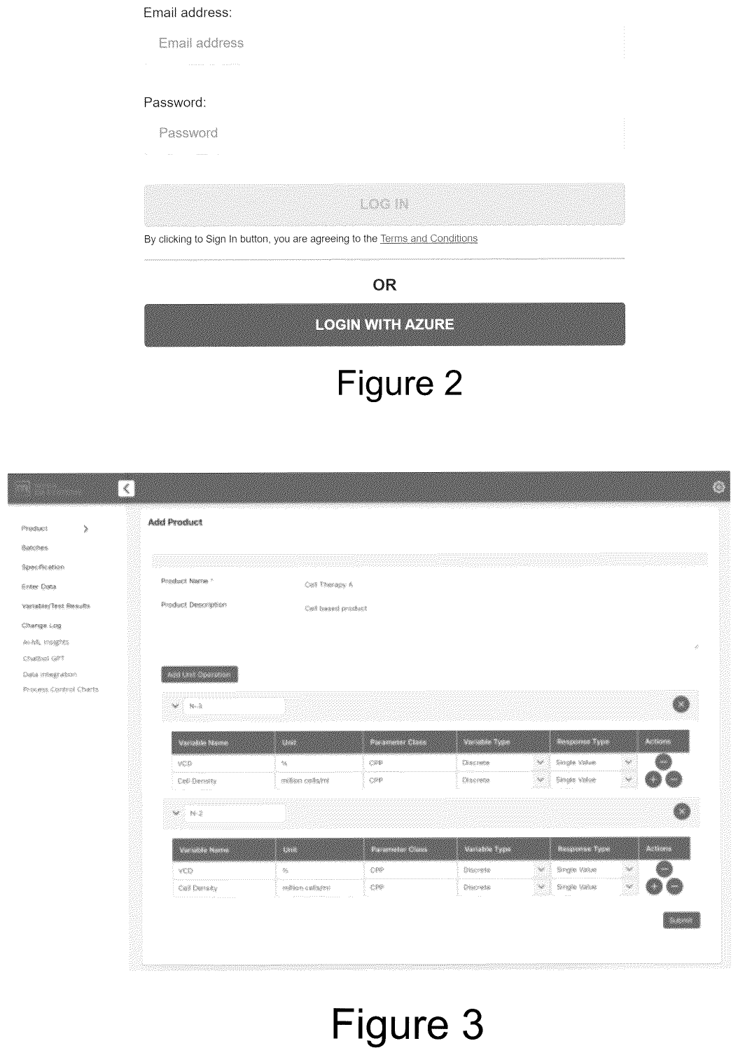

FIG. 2 shows a login interface that the user can input their email address and password. The login could be an email address, user ID, name, or code, while the password could be biometric like a fingerprint, iris, or face scan, voice imprint, password, pin number, or any other identifying mode.

FIG. 3 shows the interface enabling the user to input product information to track via the system, which allows the user to input the information shown to track the product in the supply chain. While the screen shot is shown as a preferred example of the system, variations of the screen shown could be used depending on the preferences of the user. The orientation of the options on the side of the screen could be located at any point on the page, in a pull down menu, animated buttons or options, in any order, or any variation that would allow for the same information to be presented to the user. The options shown could be a default setting of the software, which can be customized by the user and saved for future use. This customization would allow for buttons, graphs, images, and any information presented to be added or deleted from this page to only show the information required by the user. The software could also store a plurality of profiles for each user so that a user's preferences could be called up by pressing a button on the page or during a login screen presented when the software is started, allowing for the current user to enter their login information and/or password, select a stored profile, create a new profile, or select the default UI to save time. The profile or preferences could be used for each selectable window, or the profile or preferences could be used for every selectable window available while using the software. The default display could display a line graph as shown, any type of graph or visual representation of the information requested could be used, such as bar graphs, pie charts, 3D graphical representations, raw data, or any other means for displaying information to a user. While this description is applied to FIG. 1, the description would be applicable to every window discussed in the application. The actions button can used be adding information needed for the inventory analysis via a plus sign icon, minus sign icon, pencil icon, or any selectable means for bringing up additional menus.

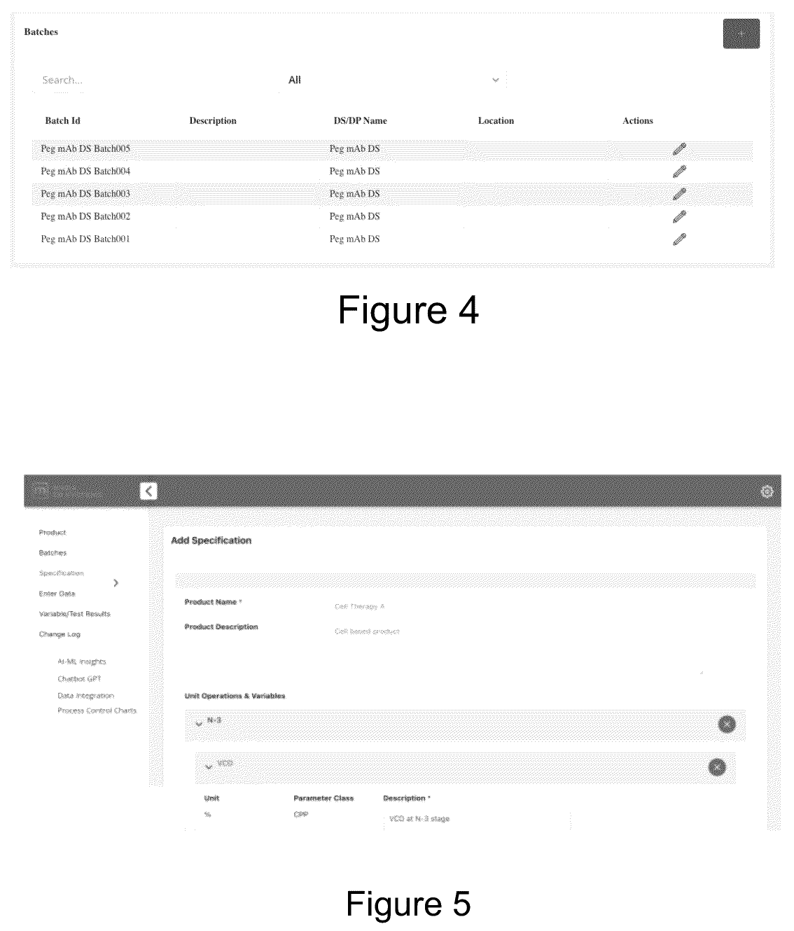

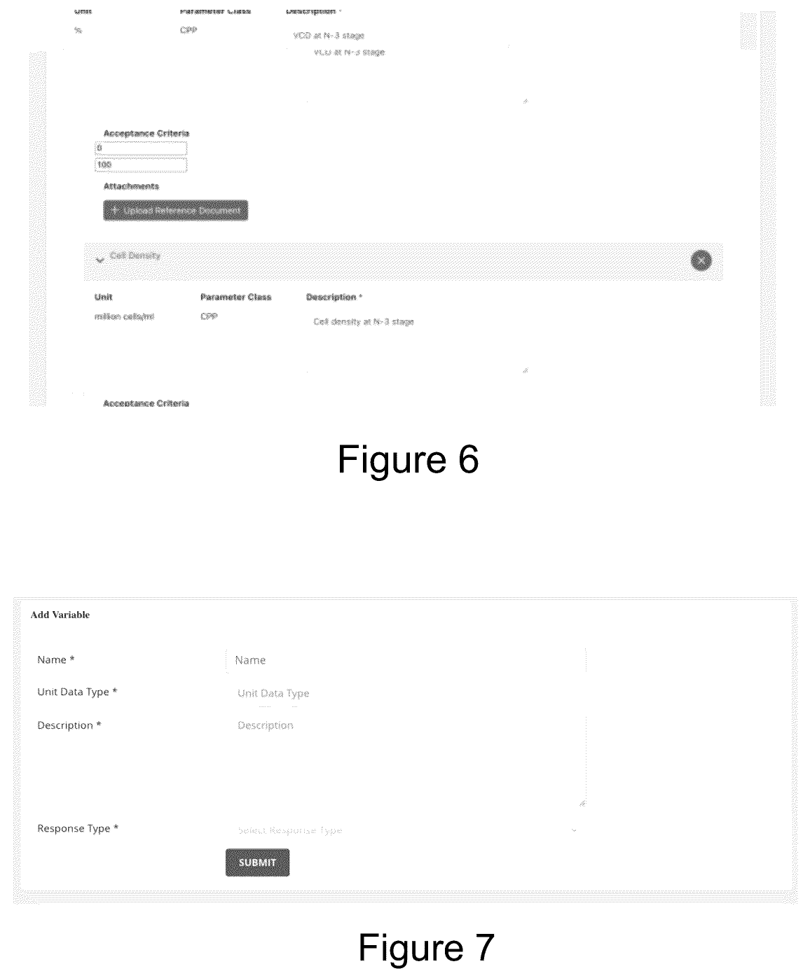

FIG. 4 shows the user interface that allows the user to input the batch information for each product. FIG. 5 shows the user interface for inputting the information for a particular product. FIG. 6 shows the user interface that allows the user to input the Acceptance Criteria, which would include shelf life of the pharmaceutical or compounds being used. The interface also allows for the user to upload a Reference Document that would automatically fill in the required fields without requiring the user to input the information manually. FIG. 7 shows the user interface for adding variables for each pharmaceutical that could affect the shelf life of the pharmaceutical. In this interface the user can input unit data types and/or unit operations (such as protein A or bioreactor) for the variables.

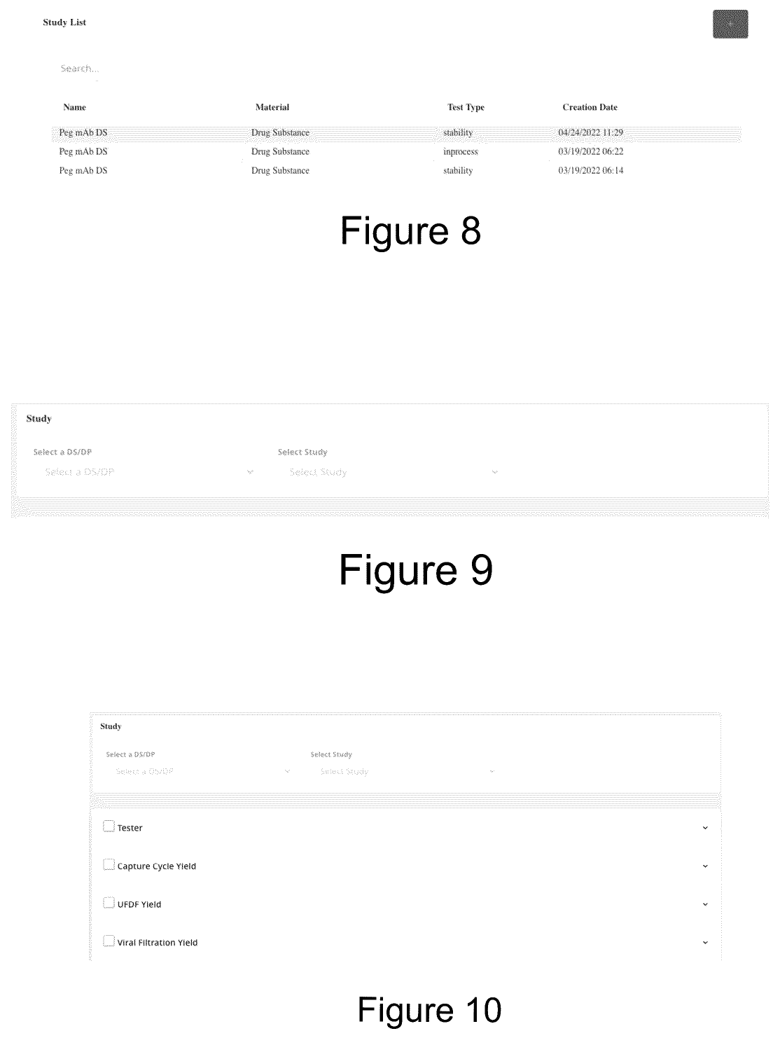

FIG. 8 shows a user interface where information about clinical batch can be inputted into the system. FIG. 9 shows the interface for inputting the study information. FIG. 10 shows the interface for entering in the specifics for a study/batch, which allows the user to select a plurality of variables that have already been entered into the system by the user or another user to select what the study measures.

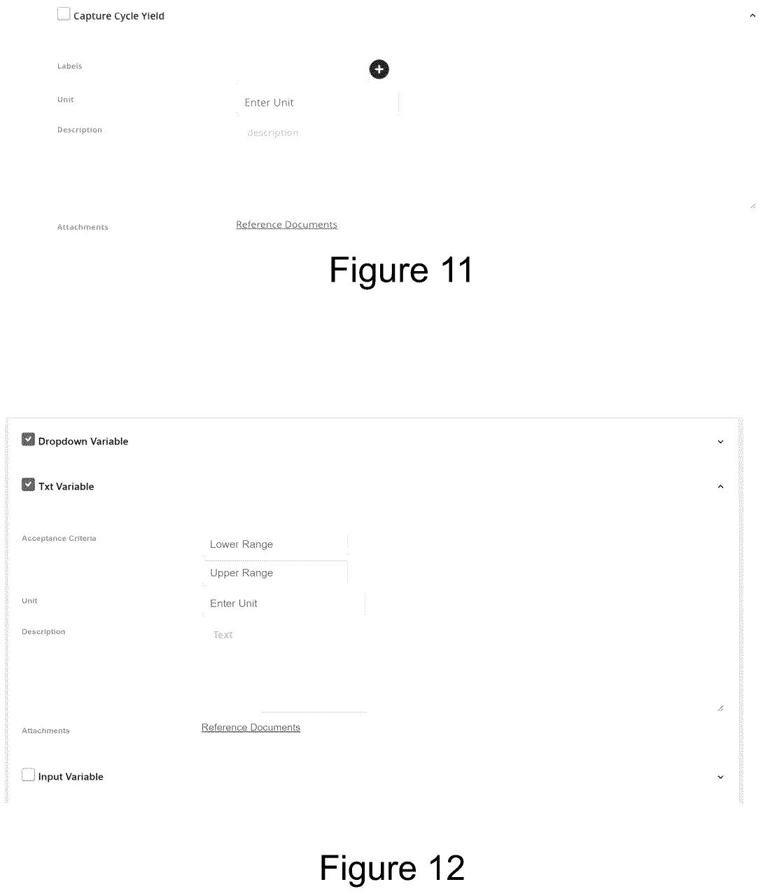

FIGS. 11 and 12 show the user interface for entering Capture Cycle Yield variable (example)information, and by selecting the variable name and whether to include it in the current study. That way the user can customize the parameters that are then viewed by the user.

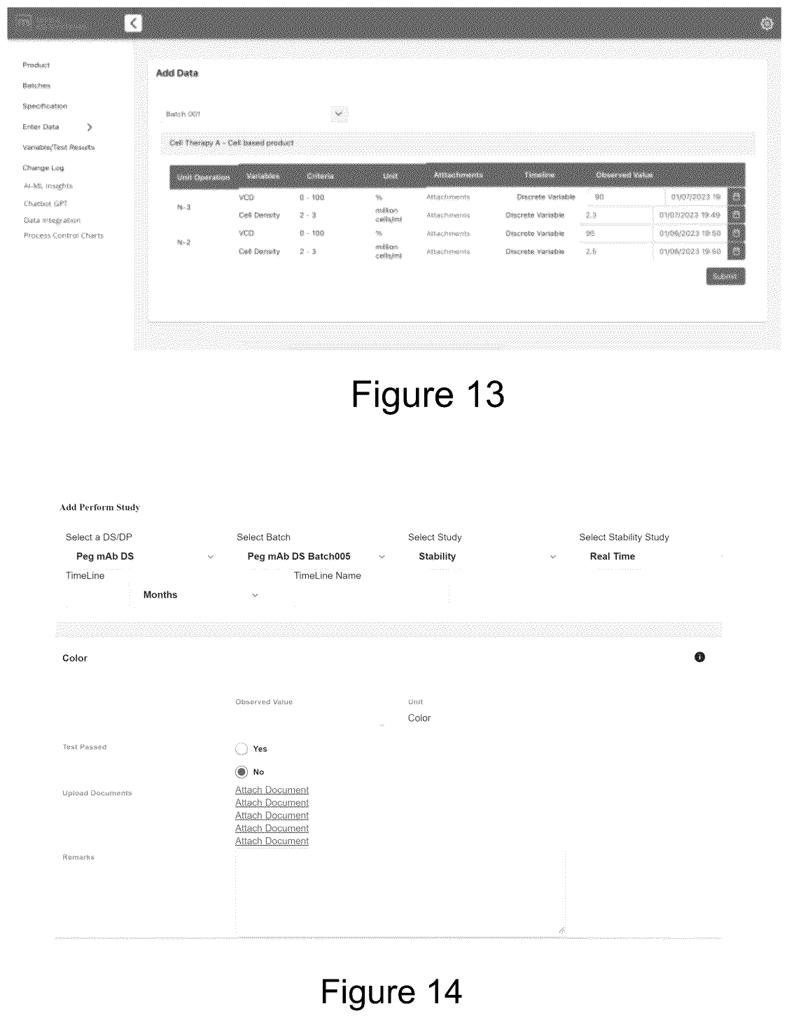

FIGS. 13 and 14 show the user interface for inputting new data entries by selecting from the drop down (or any input method) menus, which allow the user to select pharmaceuticals, batches, studies, and timelines. In the case of process data where the timeline is not relevant, such as during process yield, the user can leave the timeline field empty. There is also information pertaining to the color of a pharmaceutical, including tests passed, the observed color value, remarks, and upload documents that could be used in the stability determinations.

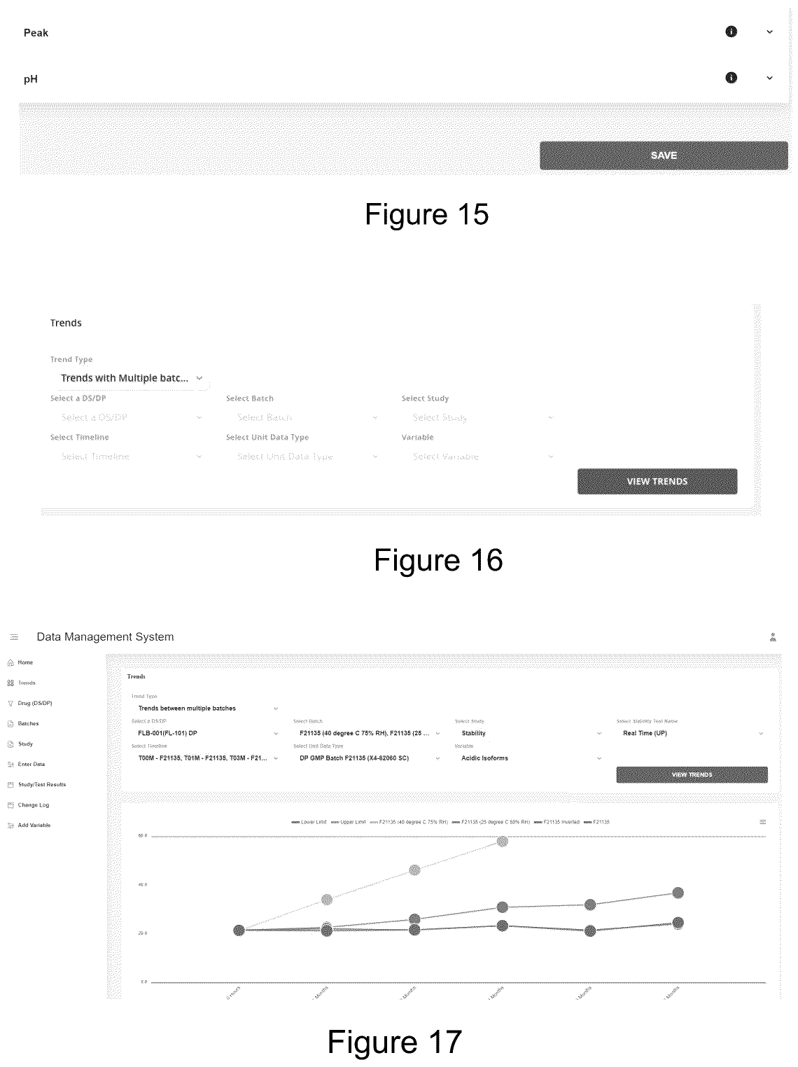

FIG. 15 shows where additional information can be inputted, such as Peak values and pH levels. Once all the information is entered in by the user, the user can select the SAVE option to save the information into the system. FIG. 16 shows the user interface allowing for the user to select the different trends in the Stability data generated by the system. FIG. 17 shows a line graph depicting the trend lines for each compound and/or pharmaceutical that the user requests to view the stability information. The user can select trends within a batch, which allows the user to input multiple studies within the same batch in order to create a visual representation of how a study affects the variable across the same batch. Trends between multiple batches can also be selected, which allows the user to input multiple batches and a single study in order to create a visualization of how the study affects the variable across different batches. Trends with multiple batches without timeline can also be inputted, which allows the user to input multiple batches across a single timeline and single study. This will allow the user to create a visualization of how the variable affects across different batches

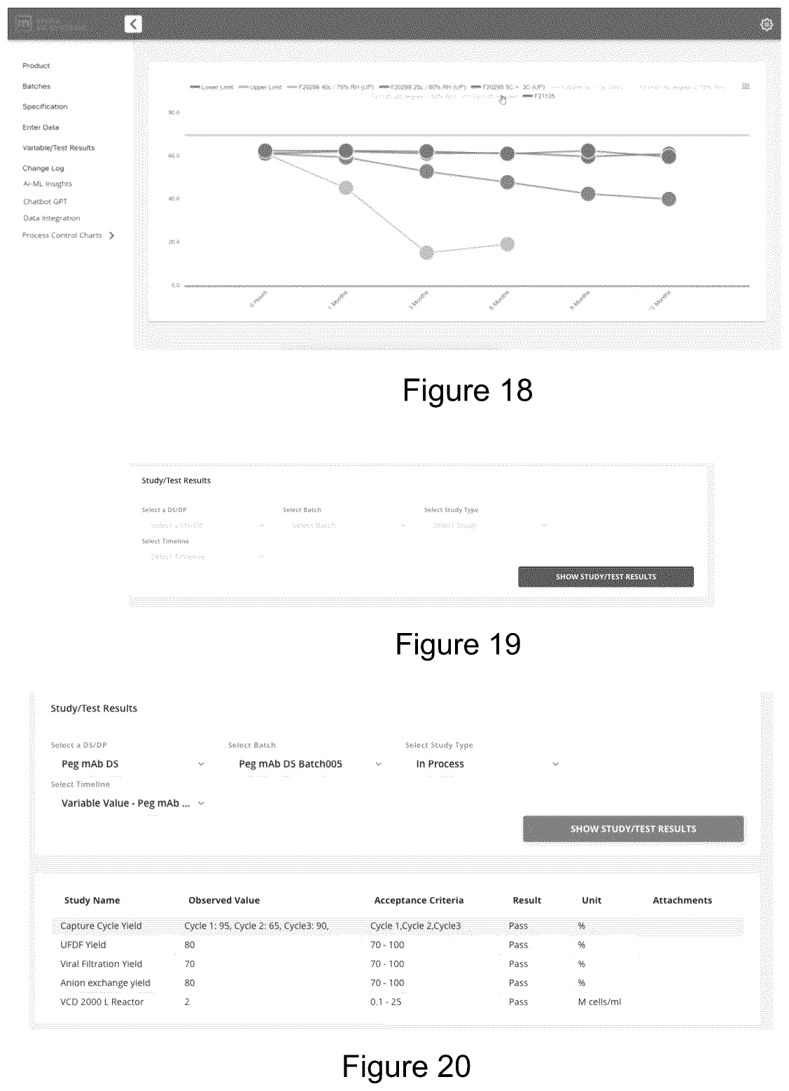

FIG. 18 shows the output of the information requested by the user, which is presented as a line graph, but any type of graph, visual representation, or text could be used to present the information to the user. FIG. 19 shows the user interface which allows for the user to view any study and/or test results data which has been inputted into the system. FIG. 20 shows addition drop down menus for selecting the desired study and/or test results.

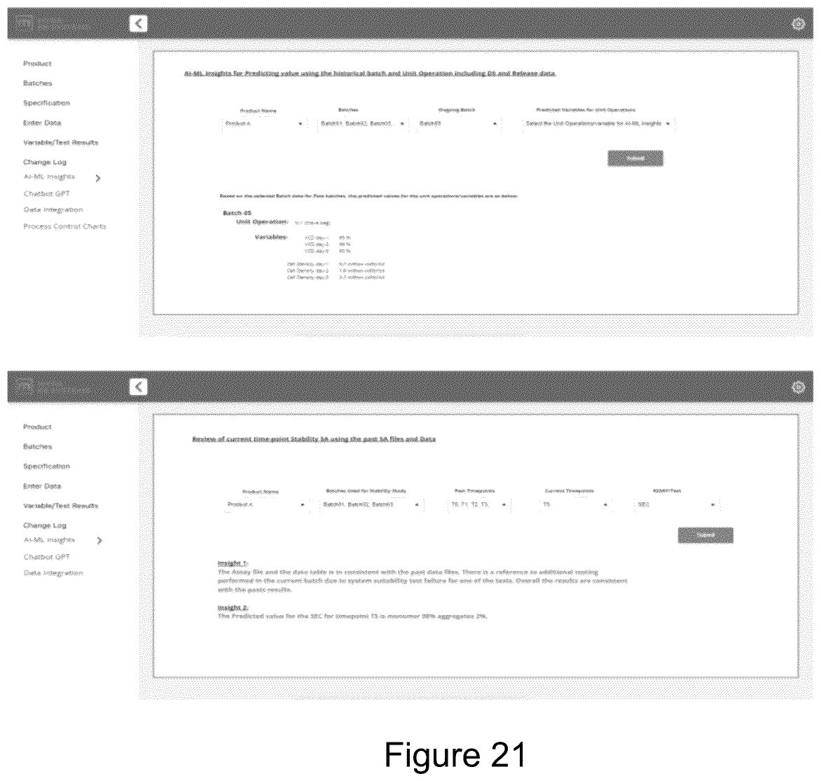

FIG. 21 shows the interfaces for manipulating the aggregated and generated data using the AI and ML tools. The tools is used to create insights into the stability for a particular pharmaceutical. The tools can use a system like ChatGPT-3 (or any conversational AI chat program) to ask real language questions for the system and output the information to the user in an easy to read and interpret the large amount of data aggregated and created using the system.

The devices mentioned above could be implemented using any type of processor architecture able to execute software including, but not limited to, x86, ENIAC, RISC, Pentium™, and Apple Silicon™. The software could be any type of code that is used to instruct a processor to perform instructions including, but not limited to, Python™, Java™ C+™ FORTRAN, and Assembly. The software could be stored on any type of non-transitory medium including, but not limited to, RAM, ROM, Flash Memory, SD cards, solid stated drives, spinning platter storage devices, Punch Cards, Piano Player Reels, Hard Drives, and physical servers.

We claim:

1. A system for determining pharmaceutical stability based on collected and aggregated data in the application, comprising:
an interface allowing a user to input information to a pharmaceutical batch for a time window, where the information to include into the batch information is captured from outside databases;
the system analyzes the information captured and any information attached by the user for the batch, and the user can input control limits including inputted allowed standard deviations;
using the captured information, the attached information, and the inputted control limits for a pharmaceutical batch, the AI or ML system can aggregate the data used in the pharmaceutical creation process and generate control charts and stability trends for the batch;
an interface to allow for the user to select different and change parameters to determine how the project stability information changes based on the parameter changes and display the change to the user.

2. A system for determining pharmaceutical stability based on collected and aggregated data in the application as claimed in claim 1, further comprising:
the data extracted is data from a PI system, LIMS lab system, ELN, or Excel templates.

3. A system for determining pharmaceutical stability based on collected and aggregated data in the application as claimed in claim 1, further comprising:
the attached files relate to Release and Stability data.

4. A system for determining pharmaceutical stability based on collected and aggregated data in the application as claimed in claim 1, further comprising:
wherein based on Bio-Reactor data, the system can predict yield data at later steps in the pharmaceutical manufacturing.

5. A system for determining pharmaceutical stability based on collected and aggregated data in the application as claimed in claim 1, further comprising:
wherein the time window is a month or year.

6. A system for determining pharmaceutical stability based on collected and aggregated data in the application as claimed in claim 1, further comprising:
wherein the changes are displayed via a visual representation.

7. A system for determining pharmaceutical stability based on collected and aggregated data in the application as claimed in claim 6, further comprising:
wherein the visual representation is a natural language text response.

8. A system for determining pharmaceutical stability based on collected and aggregated data in the application as claimed in claim 1, further comprising:
wherein the visual representation is a bar graph, line graph, or chart.

9. A system for determining pharmaceutical stability based on collected and aggregated data in the application as claimed in claim 1, further comprising:
wherein the AI or ML system uses natural language processing to process input from the user and output the information in a natural language output to the user.

10. A system for determining pharmaceutical stability based on collected and aggregated data in the application as claimed in claim 1, further comprising:
wherein the AI or ML system uses natural language processing to process input from the user and provide details on stability analysis by reviewing all the attached files and output a decision whether the data is on track; and
wherein the ML system analyzes acceptance criteria and data the ML is trained on including review data and attached files, using the analysis to output a stability trend snapshot to the user.
